(19) United States
(12) Patent Application Publication
(10) Pub. No.: US 2026/0165649 A1
(43) Pub. Date: Jun. 18, 2026

(54) SYSTEMS AND METHODS FOR MONITORING HEALTH STATUS AND LOCATION OF A SUBJECT

(71) Applicant: Michelle Noél Falbo, Katy, TX (US)

(72) Inventor: Michelle Noél Falbo, Katy, TX (US)

(21) Appl. No.: 19/186,338

(22) Filed: Apr. 22, 2025

Related U.S. Application Data
(60) Provisional application No. 63/735,434, filed on Dec. 18, 2024.

(51) Int. Cl. A61B 5/00; A61B 5/0205; A61B 5/11

(52) U.S. Cl. CPC A61B 5/6802; A61B 5/0022; A61B 5/0205; A61B 5/1112; A61B 5/746; A61B 2560/0242; A61B 2560/0266

(57) ABSTRACT

A system for monitoring health status and location of a subject with a wearable device including: a sensor to collect body parameter data, a sensor to detect tampering, and a GPS module. A processor is coupled to the body parameter data sensor, the tampering detection sensor, and the GPS module, and configured to process: data collected by the body data sensor, a signal from the tampering detection sensor, and GPS data. The processor: determines, based on the processed data collected by the body parameter sensor, if a predefined data parameter is recognized; activates an alert if the recognized data parameter meets a predefined criterion; activates an alert if the tampering detection sensor outputs the signal; and activates an alert if the device enters or exits a predefined geographical zone based on the processed GPS data. A computer-implemented method for monitoring health status and location of a subject.

### CROSS REFERENCE TO RELATED APPLICATIONS

This patent application claims the benefit of priority of US provisional patent application Ser. No. 63/735,434 filed on Dec. 18, 2024.

### BACKGROUND

### Field of the Invention

The present disclosure relates generally to systems and methods for monitoring health status and location of a subject. More specifically, the present disclosure relates to a wearable device for monitoring health status and location of a subject and a system for providing notices and alerts to designated parties in real time.

### Background of the Related Art

Increased concern over health care has led to a wide variety of health monitoring devices. Conventional health monitoring devices are able to monitor the medical conditions of an individual using sensors that provide an indication of the person's body parameters. However, such devices are generally limited to specifically targeted health monitoring, such as an individual's heart rate or blood sugar level. Conventional devices and systems are also limited in the scope of notice provided when an abnormal health condition is detected.

### BRIEF SUMMARY

Some embodiments provide a system for monitoring health status and location of a subject including a wearable device configured to be placed on the subject. The device includes at least one sensor to collect body parameter data from the subject, a sensor to detect tampering with the device, and a Global Positioning System (GPS) module. A processor is communicatively coupled to the at least one body parameter data sensor, the tampering detection sensor, and the GPS module. The processor is configured to process various data and signals, including data collected by the at least one sensor, a signal output from the tampering detection sensor, and data from the GPS module. The processor is further configured to activate a health alert if at least one body parameter meets a predefined criterion, activate a tampering alert if the tampering detection sensor outputs the signal, and activate a location alert if the wearable device enters or exits a predefined geographical zone based on the processed data from the GPS module.

Some embodiments provide a computer program product and/or a computer-implemented method for monitoring health status and location of a subject that includes receiving, via a communication network, data collected by a wearable device placed on a subject, wherein the device includes at least one sensor to collect body parameter data from the subject, a sensor to detect tampering with the device, and a Global Positioning System (GPS) module. A processor that executes program instructions of the computer program product will process data received from the wearable device, including the data collected by the at least one sensor, a signal output from the tampering detection sensor, and data from the GPS module. The processor also transmits an alert, via the communication network, to a remote computing device if the recognized at least one data parameter meets a predefined criterion, if the tampering detection sensor outputs the signal, and/or if the wearable device enters or exits a predefined geographical zone based on the processed data from the GPS module.

### BRIEF DESCRIPTION OF THE SEVERAL VIEWS OF THE DRAWINGS

The following figures form part of the present specification and are included to further demonstrate certain aspects of the present disclosure and should not be used to limit the claimed subject matter. A more complete understanding of this disclosure and the features and advantages thereof may be acquired by referring to the following description taken in conjunction with the accompanying drawings, in which like reference numerals may identify like elements. It should be understood that the disclosed embodiments are not limited to the precise arrangements and configurations shown in the figures. Also, the figures are not necessarily drawn to scale, and certain features may be shown exaggerated in scale or in generalized or schematic form in the interest of clarity and conciseness.

FIG. 1 is a perspective view of a wearable device system according to an example embodiment of this disclosure.

FIG. 2 is a plan view of a wearable device system according to an example embodiment of this disclosure.

FIG. 3 is a partial exploded view of a wearable device system according to an example embodiment of this disclosure.

FIG. 4 is a perspective view of a wearable device system according to another example embodiment of this disclosure.

FIG. 5 is a schematic diagram of a system including a wearable device, where the system is integrated in a communication network according to an example embodiment of this disclosure.

FIG. 6 is a schematic diagram of a system including a wearable device, where the system incorporates an artificial intelligence engine according to an example embodiment of this disclosure.

FIG. 7 is a schematic diagram of a software as a service (SaaS) model incorporating a system that includes a wearable device according to an example embodiment of this disclosure.

### DETAILED DESCRIPTION

While various embodiments are described herein, in the interest of clarity all features of an actual implementation may not be described in this specification. In the development of any such actual embodiment, numerous implementation-specific decisions may need to be made to achieve the design-specific goals, which may vary from one implementation to another. It will be appreciated that such a development effort, while possibly complex and time-consuming, would nevertheless be a routine undertaking for persons of ordinary skill in the art having the benefit of this disclosure.

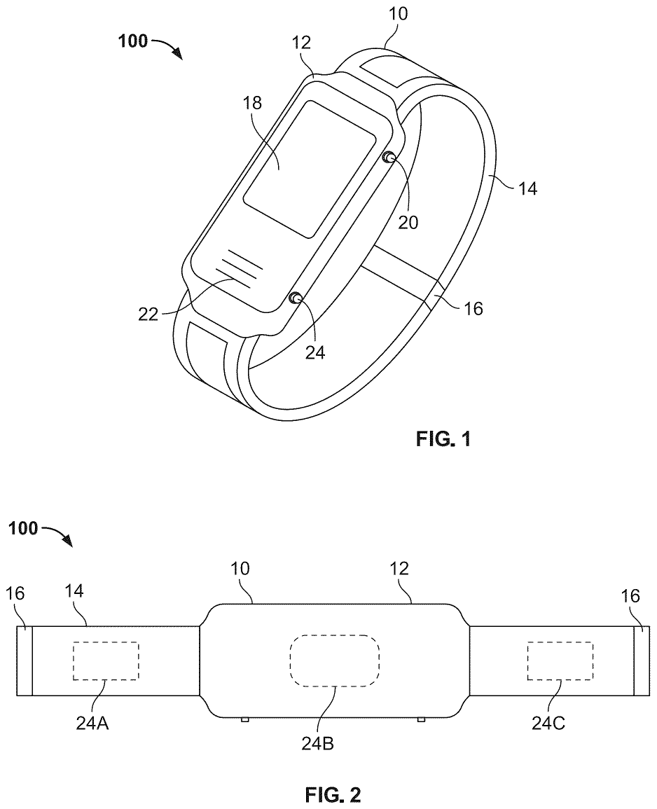

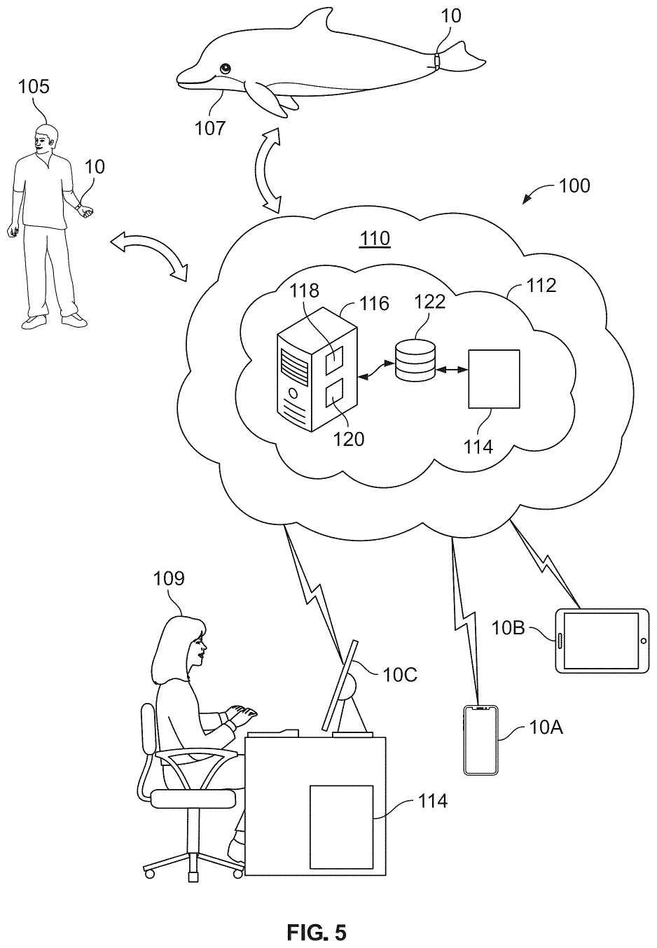

FIG. 1 is a wearable device 10 according to an example system 100 embodiment of this disclosure. The device 10 includes a case 12 with a band 14 operable to be placed on a subject (see FIGS. 5, 105, 107). In some embodiments, the wearable device 10 is akin to a wristwatch that can be worn on the wrist by a subject. The band 14 can be of any length and width as desired depending on the application. Useable materials for the band 14 include soft, flexible, durable, non-toxic, and hypoallergenic materials. Some bands 14 are made using water resistant materials as known in the art (e.g., silicone based materials, cut-resistant thermoplastic polyurethane, etc.). Embodiments include a locking mechanism 16 to prevent accidental removal of the device 10. The locking mechanism 16 can be implemented using mechanical interlocks, including for example, magnetic closures, as known in the art. Devices 10 may also be implemented with conventional smart locking mechanisms 16 that require authentication (e.g., via a mobile app) to lock and unlock.

In some embodiments, the locking mechanism may be omitted. For example, a wrist band with a conventional buckle or clasp closure, magnetic closure, hook and look fastener type closure and the like may be used in combination with a tamper detection sensor, which could be referred to as a removal sensor. The removal sensor would detect when the wearable device is taken off or removed from the subject, such as when the removal sensor (such as one or more of the sensors 24A, 24B, 24C described below) detects there is a loss of contact with the skin or when the removal sensor detects separation of two elements of the wrist band. Optionally, the removal sensor may also be used to detect a body parameter but will sense removal from the subject when the body parameter is no longer detectable. In these embodiments, the focus is on detecting that the wearable device has been removed rather than preventing the wearable device from being removed.

The case 12 is configured with a display surface 18. Some embodiments of the device 10 may be implemented with an Organic Light Emitting Diode (OLED) display surface 18. The display surface 18 can be configured to remain off to save power and activate when an alert is triggered or upon touch of the display. Embodiments of the wearable device 10 may also be made completely waterproof. Some embodiments of the device 10 may be implemented with a communication button 20 and a transceiver portal 22 to provide for two-way wireless communication (further described below). Embodiments may also be implemented with a data button 24 to allow the user to toggle through various information sets (e.g., subject's name, emergency contact phone number, battery status, etc.) for display on the OLED display surface 18. The stored/viewable information sets can be programmed as desired (further described below).

FIG. 2 is a schematic plan view of another wearable device 10 embodiment. The device 10 is shown with the display surface 18 face down, revealing the inner side of the case 12 and open band 14. As shown in FIG. 2, embodiments may be implemented with one or more sense pads or sensors 24A, 24B, 24C disposed on the case 12 and/or the band 14. The types of sensors 24A, 24B, 24C that can be implemented with device 10 embodiments include, but are not limited to, conventional: photoplethysmography sensors (measure heart rate and blood oxygen saturation); electrocardiogram sensors (measure heart rate); temperature sensors (measure skin and/or ambient temperature); and bioimpedance sensors (measure hydration and stress levels). It will be understood that other conventional sensors, and sensors not yet developed, may be used with implementations of the disclosed systems 100.

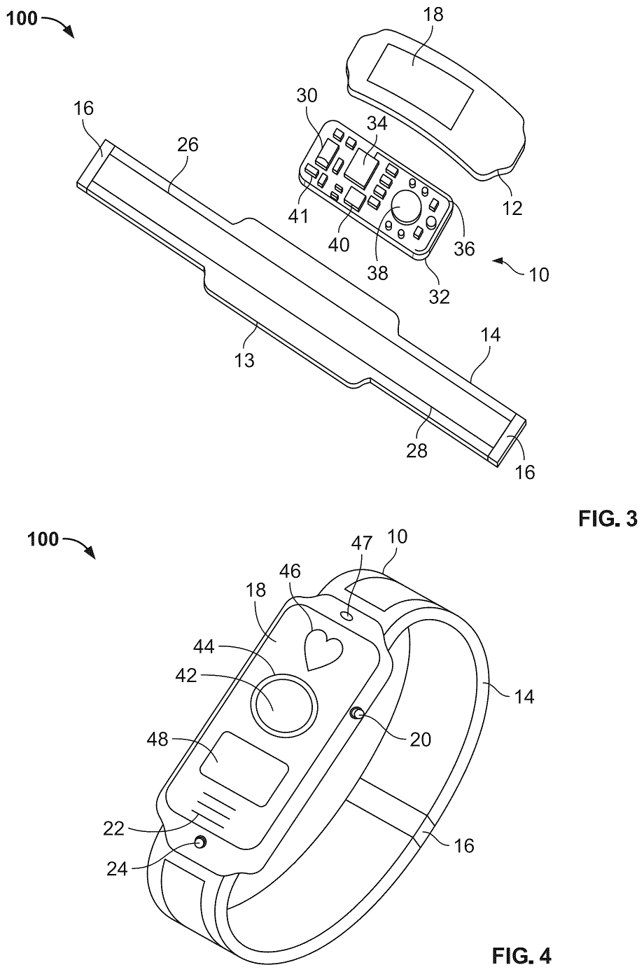

FIG. 3 is a partially exploded view of a wearable device embodiment 10. Some embodiments may be implemented with a tampering detection sensor 26 built into the device. FIG. 3 shows a tampering detection sensor 26 implemented with a conductor 28 running along the band 14, from one end to the other and through the case 12 section. When the device 10 is secured on a subject, any tampering or attempt to cut the band 14 or break the case 12 will affect the conductor 28, which will trigger an automatic alert (further described below) and a high-pitched (e.g., 90-decibel) alarm via a transceiver 30 disposed on a circuit board 32 contained within the case 12. In some embodiments, the tampering detection sensor 26 is linked to the locking mechanism 16 (see FIG. 1) to trigger the alarm and alert if the band 14 is opened, unclasped or otherwise removed without authorization. Wearable device 10 embodiments may be configured with other sensors as known in the art to implement the tampering detection sensor 26 (e.g., pressure sensors to detect excessive stress applied to the band, proximity sensors to detect an unintended break in the band connection, etc.).

The circuit board 32 includes conventional circuitry and electronics to perform the functions of the disclosed device 10 embodiments. For example, a GPS module 34 to provide real-time location tracking, an antenna 36, battery 38, microprocessor 40, an accelerometer/gyroscope 41, etc. Other components may include a security and encryption module (ensures secure data transmission) and a real-time clock module (accurate time tracking). FIG. 3 shows an embodiment wherein the case 12 is separable from a base section 13. Other embodiments may be implemented with the case 12, the base 13, and the band 14 formed as a single unit (i.e., a silicone, plastic, or composite enclosure formed via fabrication techniques as known in the art).

FIG. 4 shows another embodiment of the wearable device 10 of this disclosure. This embodiment includes an emergency alert button 42 disposed on the display surface 18. The button 42 can be disposed on the display surface 18 as shown in FIG. 4, or in another position on the case 12. This button 42 provides the user with a quick and easy way to trigger an alert (further described below) by simply depressing the button when danger is perceived. Some embodiments of the device 10 may also be configured to trigger the high-pitched alarm via the transceiver 30 (see FIG. 3) when the button 42 is depressed. Embodiments may be implemented with a hinged protective cover or cap 44 to prevent accidental activation of the emergency alert button 42. Some embodiments of the device 10 may also include a warning Light Emitting Diode (LED) 46, which is set to flash when a predefined parameter or set of parameters is recognized from the data collected from the sensors 24A, 24B, 24C. The LED 46 can be configured to transition from one color to another (e.g., blue to red) to indicate a changing health condition from a dangerous to critical zone (further described below).

FIG. 4 also shows an embodiment with a biometric input sensor 48. The sensor 48 can be any conventional sensor or sense pad as known in the art for detecting biometric parameters. Wearable device 10 embodiments may be implemented with a biometric sensor 48 configured to detect fingerprints, facial recognition, retina patterns, etc. For example, the onboard microprocessor 40 (see FIG. 3) can be programmed to only permit access, control, and modification of the device's 10 features to individuals whose fingerprints are initially programmed into resident memory upon activation of the device. The device 10 can be programmed to only permit individuals recognized via the detected biometric parameter(s) to unlock the locking mechanism 16 and remove the device 10 from a subject. Tampering or unauthorized unlocking of the locking mechanism 16 will trigger the alarm and alert as described herein. The biometric sensor 48 can be mounted on the display surface 18 as shown in FIG. 4 or in another position on the case 12 or the band 14.

FIG. 5 shows another embodiment of the system 100 of this disclosure. It will be appreciated that the wearable devices 10 may be used in applications for humans as well as with wildlife. FIG. 5 shows devices 10 worn by a human being 105 and a sea creature 107. For purposes of this disclosure, both humans and wildlife are referred to herein as “subjects.” As shown in FIG. 5, the device 10 is placed on the subject 105, 107 such that the sensor(s) 24A, 24B, 24C (see FIG. 2) can collect body parameter data from a selected place on the subject's body.

As shown in FIG. 5, the wearable devices 10 can be communicatively linked to a communication network 110 that provides communication links between one or more computing devices such as a mobile smart phone 10A, a tablet computer 10B, and a desktop or laptop computer 10C. The computing devices may be any conventional computer devices equipped with a visual display, telecommunication circuitry, and/or voice-command ability. The communication network 110 may be the Internet, an intranet, a wired or wireless network, Bluetooth, a Wi-Fi network, a cellular network, a satellite network, or any combination thereof. The system 100 includes system architecture 112 including an application program 114 (including an application programming interface), a server 116 configured with one or more microprocessors 118 and a memory module 120 (transitory and non-transitory). Some embodiments may also include a database 122. The architecture 112 may be implemented as a unitary structure (e.g., central server at a main site) or as a cloud-based architecture. Use of the term “cloud” in this context refers generally to conventional cloud computing, which is a paradigm of computing in which dynamically scalable and often virtualized resources may be provided as a service over the network 110.

The software constructs enabling the embodiments of this disclosure may reside in the application program 114 of the system architecture 112 and/or in the microprocessor 40 in the wearable device 10 (see FIG. 3). Embodiments of the software code of the application program may be implemented using conventional programming languages as known in the art (e.g., JAVA™, PYTHON™, C, C++, etc.). It will be appreciated by those skilled in the art that the application program 114 may be implemented with a single software program or a group of programs designed to perform the activities of the disclosed embodiments. The system architecture 112 may be implemented with conventional computer hardware (e.g., server systems) situated in one location or via a distributed cloud-based network.

Those skilled in the art will appreciate that the embodiments of this disclosure may be implemented in network 110 computing environments with many types of computer system configurations, including desktop computers, laptop computers, personal computers, message processors, hand-held devices, multi-processor systems, microprocessor-based or programmable consumer electronics, network PCs, minicomputers, mainframe computers, and/or conventional cellphones. Embodiments may be implemented in distributed system environments where local and remote computer systems, which are linked (either by hardwired data links, wireless data links, or by a combination of hardwired and wireless data links) through a network 110, both perform tasks. In a distributed system environment, the application program 114 may be located in both local and remote memory storage devices. Some embodiments of the system 100 may be implemented using conventional memory constructs (e.g., local memory, virtual memory, and/or cloud-based memory).

The communication network 110 opens the field of application and operations for the wearable devices 10 beyond the individual subject's 105, 107 use. Via the network 110, a remote party 109 is communicatively linked to the respective device(s) 10 configured for real-time interactive two-way communication. For example, a device 10 can be programmed (via any of the communication devices 10A, 10B, 10C and the resident software in the device 10) to allow the subject 105 and any number of desired parties 109 to receive alerts, update the resident device software, have two-way audio/text communication, collect and process the sensor 24A, 24B, 24C data from the device, track the location of the device, set up specific alerts depending on the detected health parameters and/or device location, send alert notices to designated devices/parties, and update/revise the viewable information sets. Some embodiments of the device 10 may also be implemented with a conventional video display integrated in the display surface 18 to provide two-way visual data streaming as known in the art.

The wearable device 10 provides an unprecedented tool to prevent, detect, combat and/or stop sex trafficking in real time. As disclosed herein, the device 10 provides GPS tracking, life-saving medical condition monitoring and alerts, biometric protection, tamper alarms, and law enforcement integration. As shown in FIG. 5, the communication network 110 links the subject 105 (e.g., a child) wearing the device 10 to an authorized party 109, such as law enforcement authorities, as well as parents, guardians, and/or other authorized parties 109. This real-time inter-party communication provides the subject 105 that is wearing the device 10 with unparalleled security. With real-time monitoring, early warning systems, and/or the ability to integrate with law enforcement systems and/or databases, the device 10 equips the authorized party(ies) to act quickly and decisively.

In one non-limiting example, if a trafficker or abductor attempts to cut or tamper with the device 10, an instant alarm and alert may be triggered, as described herein. The device's 10 sensors 16, 26, for example, may detect forceful tampering or removal attempts. The high-decibel audible alarm via the transceiver 30 may activate to alert bystanders and deter an abductor. Some embodiments of the device 10 may also be configured to automatically lock itself (i.e., physically lock the device to the subject and/or digitally lock the user interface and buttons) if tampering is detected, to prevent unauthorized reconfiguration. Automatic alert notifications may be sent instantly to the authorized party or parties 109, including law enforcement authorities (e.g., local police, FBI, and other law enforcement networks integrated into the system 100 via the communication network 110). Device 10 (and, therefore, the subject 105) location and emergency data are logged immediately via the network 110 and preferably stored in the database 122 or other storage device and data structure, allowing law enforcement to take action in real time. The biometric sensor 48 ensures only authorized individuals can access or remove the device 10 from the subject 105, thereby preventing tampering attempts, and/or ensures that only authorized individuals are permitted to alter device settings or access data stored on or accessible through the device 10. Authorized individuals may also be permitted to remotely lock and unlock the wearable device 10 using biometric authentication via an app programmed into the communication devices 10A, 10B, 10C linked into the network 110, adding an extra layer of security.

The wearable device 10 may provide real-time GPS tracking with law enforcement integration. The movements of a subject 105 wearing the device 10 can be tracked via the network 110 in real time, providing location updates via the network. If the primary GPS signal drops, the system 100 can switch to cellular triangulation for location tracking. Authorized parties 109 (e.g., law enforcement authorities) can receive real-time alerts when an abduction is detected or if the subject enters a predefined geographical zone (e.g., a known high-risk area). The automatic and autonomously triggered alerts provided by the device 10 may enable immediate dispatch of law enforcement authorities to a last known location of a subject 105. System 100 integration with local databases may be used to share the subject's 105 information across all relevant parties 109. System 100 embodiments may be configured for high-risk cases or cross-border abductions, to activate national and international networks 110 to coordinate a response across borders. Some embodiments may also be configured for the device 10 to trigger an automated Amber Alert, amplifying search efforts when necessary.

An authorized party 109 (e.g., a parent, caregiver, etc.) can create one or more safe zones or “geofences” around specific geographical areas (e.g., school, home, playground) via the network 110 linked to the wearable device 10. If a child wearing the device 10 leaves a safe zone without permission, the device may send an immediate alert to one or more authorized party 109, and law enforcement if so programmed. The system 100 can also track the mobility patterns and activities of the subject 105 wearing the device to detect movement anomalies. If a child wearing a device 10 shows irregular movements, such as unexpectedly entering a known trafficking hotspot (determined via GPS tracking) or area that is not typically visited, the system 100 can automatically send a warning to one or more designated/authorized parties 109 via the network 110 for follow-up. Schools can provide greater security for children during field trips, such as by securing a unit of the wearable device 10 on each child for temporary use as programmed by an authorized party 109.

The device 10 may be further configured with emergency alert buttons 42 provide for instant response coordination. For example, a child 105 wearing the device 10 can press the emergency alert button 42 if he or she feels in danger or needs help. The emergency alert button 42 triggers a real-time alert via the network 110, with the child's exact location conveyed to authorized parties 109 (e.g., caregivers, police, FBI). This enables children to ask for help discreetly, even in dangerous situations where speaking aloud isn't possible.

In addition to monitoring the health parameters (e.g., heart rate, body temperature, respiration, hydration level, etc.) of the subject 105 in real time, after the subject is recovered the device 10 may continue to monitor the subject's health and location to prevent re-trafficking. The collected health data can also inform rescue teams if medical care is needed immediately. A law enforcement party 109 can use data collected from multiple units of the device 10 to map trafficking routes and patterns. By analyzing movement patterns, law enforcement authorities can take action to intercept and prevent trafficking operations. The worldwide reach of the communication network 110 allows one or more law enforcement parties 109 to track and locate subjects 105 wearing the device 10 across state or national borders.

In addition to monitoring one or more body (health or medical) parameters of a subject 105 in real time, the device 10 enables improved elderly care. For example, the onboard accelerometer 41 in the device 10 (see FIG. 3) can detect a fall and immediately alert an authorized party 109, such as a caregiver or emergency contact. An authorized party 109, such as a caregiver, can monitor an elderly subject 105 remotely, detect health emergencies and receive automatic alerts via the communication network 110. A subject 105 suffering from dementia, for example, can be quickly located via the GPS module 34 of the device 10 if the subject wanders off unattended.

Medical responders 109 can monitor the vital signs of a subject 105 in real time via the network 110. Subjects 105 with chronic conditions (e.g., asthma, epilepsy, etc.) can be monitored 24/7 in real time. Embodiments of the device 10 may also be configured to display text messages on the display surface 18, and to emit an audible sound and/or vibrate when a message is received. For example, if an asthmatic child 105 wearing the device 10 experiences an oxygen level drop, the sensors 24A, 24B, 24C detect the drop and an alert is automatically sent to the parent 109, who can then verbally and/or via text communicate with the child to ensure the child takes preventative action (e.g., using an inhaler) to avoid an emergency situation. A subject 105 can also trigger an immediate response via use of the emergency alert button 42 and two-way voice communication via the transceiver 30 on the device 10. When placed on an infant 105, the device 10 can help prevent sudden infant death syndrome (SIDS) by monitoring the infant's vitals (e.g., heartbeat, breathing pattern/rate) and sending an immediate alert to the caregiver or parent 109 in the event that an emergency condition is detected.

Health parameters of an athlete 105 can be monitored in real time with the device 10. Coaches and trainers 109 can monitor an athlete's 105 vitals via the display on the linked computing devices 10A, 10B, 10C. During practice or events, the device 10 can autonomously monitor an athlete's 105 health condition to help prevent cardiac events, heat stroke, and overexertion. A subject's 105 stress level can be measured indirectly through the measured heart rate variability. If the microprocessor 40 processes a recognized parameter collected by a sensor 24A, 24B, 24C and determines the parameter meets a predefined criterion (e.g., excessive heart rate, dehydration, high blood pressure, etc.), the microprocessor may activate an automatic alert as described herein, thus allowing coaches and trainers (as authorized parties 109 receiving an alert) to make real-time decisions based on the athlete's collected data.

Monitoring the health and movements of wildlife (e.g., marine life, birds, land animals) is important for conservation efforts, poaching prevention, and research on ecosystems. When attached to the subject animal 105, the wearable device 10 provides real-time location tracking, monitoring of physical conditions, and environmental interactions, allowing conservationists and other parties 109 to respond quickly to threats, optimize conservation programs, and gather data critical to ecosystem management.

The onboard GPS module 34 and communication network 110 allow authorized parties 109 to track and monitor a subject animal's 105 movement and migration patterns. Device 10 embodiments configured with external ambient temperature sensors and external pressure sensors allow parties 109 to study and log/store the environmental conditions the subject 105 is experiencing in real time. For example, when the device 10 is affixed to a dolphin 105 or other marine life, the temperature and pressure sensors 24A, 24B, 24C can detect and transmit the depth and water condition data over the network 110 for viewing by the parties 109 on their computing devices 10A, 10B, 10C. The tamper alerts (described above) provided by the system 100 allows parties 109 to immediately locate the animal 105 if a poacher attempts to remove the device 10. Embodiments of the device 10 for use with wildlife subjects 105 can be configured without certain features provided with other embodiments (e.g., without a display surface 18, emergency alert button 42, LED 46, etc.).

The geofencing capability (described above) of the system 100 allows parties 109 to configure alert notices when the subject animal 105 enters or leaves a designated geographical zone (e.g., known poaching area). For marine applications, devices 10 may also be configured with a hydrodynamic case 12 and band 14 to prevent interference with the animal's 105 behavior. Embodiments of the device 10 may also be implemented with the band 14 including suction cups, harness extensions, or other features to attach the device to the subject 105 without causing harm to the animal. Devices 10 may also be implemented with onboard memory configured to store the data from one or more of the sensors 24A, 24B, 24C for downloading at a later time depending on the application (e.g., for automatic upload to the network 110 when a marine wildlife subject 105 nears the water surface and the device 10 links to the network).

Retuning to FIG. 3, some devices 10 are implemented with an onboard battery 38 to power the device electronics. The battery 38 can be a conventional rechargeable unit (e.g., lithium-ion battery) that is connected to a charger via a waterproof magnetic coupler (item 47 in FIG. 4) on the case 12. Other devices 10 may be configured for charging the battery 38 using conventional wireless pads as known in the art. Device 10 embodiments may also be configured with the microprocessor 40 programmed to send an alert to the authorized party 109 via the network 110 and flash a battery alert icon on the display surface 18 when the battery 38 power level drops below a certain threshold. Device 10 embodiments may also be implemented with the processor 40 configured with power-saving algorithms that switch to a lower frequency of data processing under certain operational states (e.g., when device 10 movement is not detected via the accelerometer/gyroscope 41).

Some embodiments of the device 10 may be configured to trigger an automatic alert via the communication network 110 as disclosed herein and to emit a high-pitched alarm via a transceiver 30 if the device is removed from a charger without authorization (verifiable, for example, by biometric authentication via an app on the communication devices 10A, 10B, 10C or via the biometric sensor 48 on the device). The device 10 can also be configured to automatically lock itself if removed from a charger without authorization. Embodiments may also be implemented to permit an authorized party 109 to remotely pause and resume charging of a device 10, or completely lock the device, using a computing device 10A, 10B, 10C linked to the network 110.

Embodiments may also be configured with solar cells integrated into the display surface 18 or the case/band to charge the battery 38. Other device 10 embodiments may be implemented with conventional automatic mechanical movements that store energy when the subject 109 moves and release the energy to power the mechanism, as known in the art (e.g., as used with battery-free wristwatches). It will also be appreciated that device 10 embodiments may be implemented with wave energy converters (WEC) to provide the power for the device. Conventional WECs are too large and bulky for many applications of the device 10. However, it will be appreciated that as WECs are miniaturized such power sources may be implemented with devices 10, particularly for use with marine wildlife subjects 105. Some embodiments of the device 10 may be configured for thermoelectric energy harvesting as known in the art. Devices 10 may be implemented with a conventional thermoelectric generator to convert body heat into energy, providing a secondary power source when the subject 105 is stationary. Any suitable energy sources as known in the art can be integrated with the disclosed systems 100 for power management. These energy sources can charge a small internal battery 38 or supercapacitor, ensuring a steady power supply for the GPS module 34 and sensors 24A, 24B, 24C. The multi-energy implemented systems 100 can ensure that a device 10 operates indefinitely without the need for battery 38 replacement or recharging.

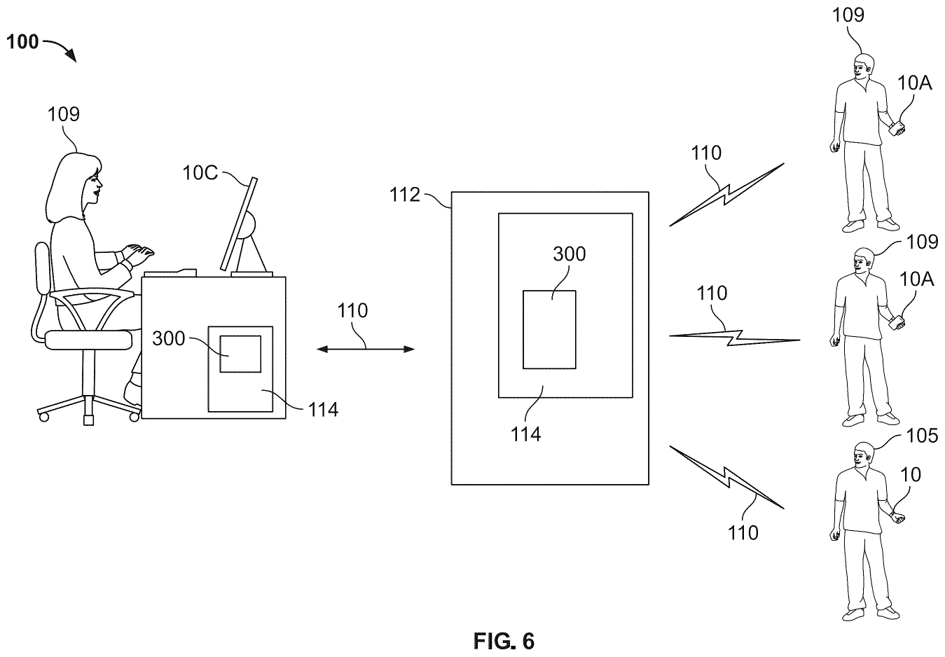

FIG. 6 shows another embodiment of the system 100 of this disclosure. A system 100 is shown incorporating an artificial intelligence engine (AIE) 300. The AIE 300 may be linked with the interface 114 or linked into the system 100 architecture 112 via the communication network 110. The AIE 300 may be configured to perform autonomous tasks on the data collected from the sensors 24A, 24B, 24C of the device 10 and the activated alerts put out by the device. For example, the AIE 300 may be configured to generate tailored notices/alerts to subjects 105 and/or authorized parties 109. The AIE 300 can trigger the transition of the LED 46 from one color to another (as described above) to indicate a changing health condition from a dangerous to critical zone based on tracked data of the subject 105 wearing the device 10. The AIE 300 can automatically detect the language of the subject 105 wearing the device 10 and provide selected translated audio/text if desired by the authorized party 109 in the event the subject speaks a different language than the party. The AIE 300 can also be configured to prioritize received alerts (e.g., for children, to dispatch the nearest law enforcement unit to the device's 10 location in immediate response).

The AIE 300 can process the received data every few seconds or at set intervals and run tailored algorithms to detect anomalies in the subject's 105 vitals to alert the subject and medical personnel 109 in the event of an emergency condition. The AIE 300 can monitor the subject's 105 overall health and identify irregularities (e.g., irregular sleep patterns, cardiac arrhythmia, etc.). For wildlife subject 105 applications, in addition to monitoring the animal's vitals, the AIE 300 can be configured to analyze the collected sensor data to identify environmental hazards or changing conditions that may be detrimental to the species. The subject 105 and/or authorized party 109 can program the AIE 300 to trigger an alert if an individual's predefined set of health parameters or parameter thresholds are detected by the device 10 sensors 24A, 24B, 24C, as custom-tailored for the subject 105. The AIE 300 can be configured to autonomously generate and send reports (e.g., via email, facsimile) to authorized parties 109, with health summaries highlighting trends and analysis of the subject's 105 vitals and health condition. This type of monitoring can help detect patterns that can lead to injuries or illness. It will be appreciated by those skilled in the art that conventional commercial artificial intelligence software may be used to implement embodiments of this disclosure. It will also be understood that artificial intelligence software, as used with device 10 embodiments, may also encompass the application of machine learning modelling.

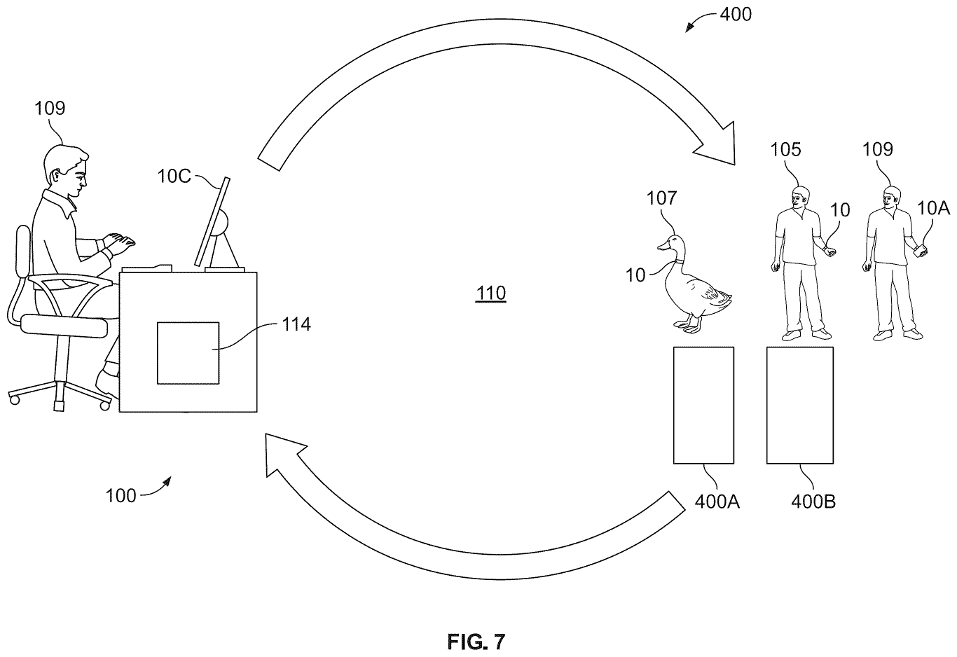

System 100 embodiments may also be implemented in software as a service (SAAS) models wherein the software is centrally hosted (e.g., on architecture 112) and remotely accessed by authorized parties 109 via the communication network 110 as disclosed herein. FIG. 7 shows a schematic of an SAAS model 400 integrating the system 100 in accordance with an embodiment of this disclosure. It will be appreciated that system 100 embodiments of this disclosure are not limited to any particular type of method or process model. In the example SAAS model 400 shown in FIG. 7, an authorized party 109 (e.g., system 100 administrator) can remotely activate, lock-unlock, send software updates, add new features, establish geofencing zones, define alert protocols, update data display formats (e.g., update dashboards viewable on computing devices 10A, 10B, 10C), generally modify settings on devices 10, and provide system 100 access to authorized parties 109 via computing devices 10A, 10B, 10C identified and linked via the network 110. In addition to the health monitoring and alert features described herein, SAAS models 400 can also be implemented to track, analyze, and keep historical data records 400A, 400B of subjects 105, 107 outfitted with a device 10. Implementation of SAAS models 400 provides an efficient and effective way to manage deployment of the devices 10, particularly in bulk deployment applications (e.g., schools, sports teams, hospitals, etc.).

By combining biometrics with geolocation and user-friendly apps on computing device 10A, 10B, 10C, the disclosed systems 100 offer comprehensive subject 105, 107 monitoring and tracking across various use cases, providing real-time insights, safety alerts, and peace of mind for authorized parties 109 such as parents. Multiple authorized parties 109 can also monitor the subject(s) 105 and receive alerts, with different levels of permission allocated to each party. Other advantages provided by the systems 100 include customizable analytics dashboards viewable on the computing devices 10A, 10B, 10C. System 100 communications and data transfer across the network 110 can also be implemented using conventional data encryption to ensure security and compliance with applicable regulations (e.g., HIPPA).

The systems 100 disclosed herein provide a versatile, wearable device 10 to track essential health metrics in real time and provide automatic/autonomous alerts and actionable insights that help prevent medical emergencies. Embodiments offer a wide range of features and benefits for children, parents, caregivers, families of the elderly, individuals managing chronic conditions, law enforcement, and others. Advantages and benefits provided by the systems 100 include: Tracking real-time heart rate and alerting users or caregivers when the heart rate is too high or too low. Detecting irregular heart rhythms, such as arrhythmia. Measuring oxygen saturation in the blood to detect respiratory issues early. Continuously monitoring body temperature and flagging sudden changes, potentially indicating fever or infection. Monitoring the subject's location in real-time. Geofencing alerts are sent to designated parties if the subject leaves a designated safe zone. Detects falls automatically and sends alerts to caregivers or emergency contacts, reducing the risk of delayed medical attention. Sets reminders for users to take their medication on time. Resistant to water, making it ideal for everyday wear and outdoor activities. All health data can be synced to a smartphone app for easy access by caregivers or family members. Providing historical data trends and insights. Early detection of health issues with continuous monitoring helps parents detect issues like fever, irregular heart rate, or low oxygen levels early. Emergency support. The SOS button empowers children to alert their parents or caregivers immediately if they feel unsafe or unwell. Wearers can send distress alerts to caregivers, family members, or emergency responders with a button press. Improved sleep patterns. Tracking sleep helps parents ensure their children are getting the rest they need for healthy development. Tracking deep, light, REM sleep and providing insights into rest quality. Proactive health management. Real-time data allows parents or caregivers to address potential health issues before they become serious. Easier medication adherence. Medication reminders ensure that children or individuals under care take their medications on time. Vital monitoring for chronic conditions. Continuous tracking of heart rate, oxygen levels, and body temperature can help manage conditions like hypertension, COPD, or diabetes. Location tracking for dementia patients. GPS tracking and geofencing provide security for elderly individuals with dementia who may wander off. Remote monitoring. Family members can monitor the health and safety of their elderly loved ones remotely through an app. Personalized health insights. Continuous monitoring offers insights into how daily activities affect health, empowering users to make informed lifestyle changes. Streamlined monitoring for multiple patients. Caregivers can monitor several individuals simultaneously using the companion app, improving efficiency. Improved communication. Health data can be easily shared with healthcare providers, allowing for better coordination of care.

As will be appreciated by one skilled in the art, embodiments may take the form of a system, method or computer program product. Accordingly, embodiments may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, embodiments may take the form of a computer program product embodied in one or more computer readable medium(s) having computer readable program code embodied thereon.

Any combination of one or more computer readable storage medium(s) may be utilized. A computer-readable storage medium may be, for example, but not limited to, an electronic, magnetic, optical, electromagnetic, infrared or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device. Furthermore, any program instruction or code that is embodied on such computer readable storage media (including forms referred to as volatile memory) that is not a transitory signal are, for the avoidance of doubt, considered “non-transitory”.

Program code embodied on a computer readable storage medium may be transmitted using any appropriate medium, including but not limited to wireless, wireline, optical fiber cable, RF, etc., or any suitable combination of the foregoing. Computer program code for carrying out various operations may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Embodiments may be described with reference to flowchart illustrations and/or block diagrams of methods, apparatus (systems) and computer program products. It will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, and/or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored on computer readable storage media is not a transitory signal, such that the program instructions can direct a computer, other programmable data processing apparatus, or other devices to function in a particular manner, and such that the program instructions stored in the computer readable storage medium produce an article of manufacture.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

It will be understood by those skilled in the art that embodiments of this disclosure may be implemented using conventional software and computer systems programmed to perform the disclosed processes and operations. It will also be appreciated that embodiments of this disclosure may be implemented using conventional and commercially available sensors, hardware, electronic components, materials, and fabrication techniques as known in the art. In light of the example embodiments described and illustrated herein, it will be recognized that numerous modifications could be applied to derive alternative embodiments of the present invention.

The terminology used herein is for the purpose of describing particular embodiments only and is not intended to limit the scope of the claims. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, components and/or groups, but do not preclude the presence or addition of one or more other features, integers, steps, operations, elements, components, and/or groups thereof. The terms “preferably,” “preferred,” “prefer,” “optionally,” “may,” and similar terms are used to indicate that an item, condition or step being referred to is an optional (not required) feature of the embodiment.

The corresponding structures, materials, acts, and equivalents of all means or steps plus function elements in the claims below are intended to include any structure, material, or act for performing the function in combination with other claimed elements as specifically claimed. Embodiments have been presented for purposes of illustration and description, but it is not intended to be exhaustive or limited to the embodiments in the form disclosed. Many modifications and variations will be apparent to those of ordinary skill in the art after reading this disclosure. The disclosed embodiments were chosen and described as non-limiting examples to enable others of ordinary skill in the art to understand these embodiments and other embodiments involving modifications suited to a particular implementation.

What is claimed is:

1. A system for monitoring health status and location of a subject, comprising:
a wearable device configured to be placed on a subject, wherein the wearable device includes at least one sensor to collect body parameter data from the subject, a sensor to detect tampering with the wearable device, a Global Positioning System (GPS) module to collect location data, and a processor communicatively coupled to the at least one body parameter data sensor, the tampering detection sensor, and the GPS module, wherein the processor is configured to:
process body parameter data collected by the at least one sensor, a signal output from the tampering detection sensor, and the location data from the GPS module;
activate a health alert if the body parameter data meets a predefined criterion;
activate a tampering alert if the signal output is received from the tampering detection sensor; and
activate a location alert if the location data indicates that the wearable device has entered or exited a predefined geographical zone.

2. The system of claim 1, wherein the body parameter data includes heart rate, body temperature, respiration rate, glucose reading, oxygen saturation, and/or blood pressure.

3. The system of claim 1, wherein the wearable device further includes a sensor to measure external ambient air temperature, external air pressure, and/or motion of the wearable device.

4. The system of claim 1, wherein the wearable device further includes a transceiver to permit communication between the subject and a remote party via a communication network and a communication button to activate the transceiver.

5. The system of claim 1, wherein the wearable device further includes an emergency alert button that causes an alert notice to be transmitted to a remote computing device via a communication network and a protective cover to prevent accidental activation of the emergency alert button.

6. The system of claim 1, wherein the wearable device further includes a biometric sensor for receiving biometric input from a person, and wherein the processor is configured to determine whether the biometric input is matches a stored biometric profile for a person that is authorized to remove the wearable device from the subject and configured to deactivate a locking mechanism securing the wearable device to the subject in response to determining that the received biometric input matches the stored biometric profile for the authorized person.

7. The system of claim 1, wherein the wearable device includes a band to secure the wearable device to the subject and configured to trigger the tamper alert in response to tampering with the band.

8. The system of claim 7, wherein the wearable device is communicatively linked to a communication network and configured to transmit a notice to a remote computing device over the communication network if the tamper alert is triggered.

9. The system of claim 1, wherein the wearable device is communicatively linked to a communication network and the processor is configured to permit an authorized remove computing device to modify, via the communication network, the at least one predefined data parameter to be recognized, the predefined geographical zone, and/or the predefined criterion.

10. The system of claim 1, wherein the wearable device is communicatively linked to a communication network and configured to transmit the data collected by the at least one sensor, the signal output from the tampering detection sensor, and the data from the GPS module to a remote computing device over the communication network.

11. The system of claim 1, wherein the wearable device is communicatively linked to a communication network configured to transmit a notice to a remote computing device over the communication network if the health alert, tampering alert and/or location alert is activated.

12. The system of claim 1, wherein the wearable device is communicatively linked to a communication network and configured to access an artificial intelligence engine over the communication network, wherein the artificial intelligence engine is configured to receive the data collected by the at least one sensor, process the received sensor data to generate a health status of the subject, and transmit the generated health status of the subject to a remote party over the communication network.

13. The system of claim 1, wherein the wearable device is communicatively linked to a communication network and configured to access an artificial intelligence engine over the communication network, wherein the artificial intelligence engine is configured to receive the data from the GPS module, process the received GPS data to provide a notice to a remote party regarding the location of the device.

14. The system of claim 1, wherein the wearable device further includes an accelerometer and a gyroscope, wherein the processor receives input from the accelerometer and the gyroscope and is configured to identify that the subject has fallen based on the input from the accelerometer and the gyroscope, and wherein the processor, in response to identifying that the subject has fallen, transmits an event alert to a remote computing device over a communication network indicating that the subject has fallen.

15. A computer program product comprising a non-volatile computer readable medium and non-transitory program instructions embodied therein, the program instructions being configured to be executable by a processor to cause the processor to perform operations comprising:
receiving, via a communication network, data collected by a wearable device worn by a subject, wherein the wearable device includes at least one sensor to collect body parameter data from the subject, a sensor to detect tampering with the wearable device, and a Global Positioning System (GPS) module to identify location of the subject, and the data collected by the wearable device includes the body parameter data, a tampering detection signal, and the location data;
identifying one or more remote computing devices that are authorized to receive health alerts, tampering alerts and location alerts for the subject;
transmitting, via the communication network, a health alert to the one or more remote computing devices in response to the body parameter data meeting a predefined criterion;
transmitting, via the communication network, a tampering alert to the one or more remote computing devices in response to receiving the tampering detection signal; and
transmitting, via the communication network, a location alert to the one or more remote computing devices in response to the location data indicating that the wearable device has entered or exited a predefined geographical zone.

16. The computer program product of claim 15, wherein receiving the data collected by the wearable device comprises receiving the data at a server or a cloud computing system.

17. The computer program product of claim 15, the operations further comprising:
submitting the body parameter data to an artificial intelligence engine configured to generate a health status of the subject based on the body parameter data;
receiving the health status of the subject from the artificial intelligence engine; and
transmitting, via the communication network, the generated health status of the subject to the one or more remote computing devices.

18. The computer program product of claim 17, wherein the body parameter data comprises at least one of: heart rate, body temperature, respiration rate, glucose reading, oxygen saturation, and blood pressure.

19. The computer program product of claim 15, the operations further comprising:
receiving a communication from one of the one or more remote computing devices, wherein the communication requests a modification to the predefined criterion and/or the predefined geographical zone;
determining whether the one of the one or more remote computing devices from which the communication is received is authorized to make modifications to the predefined criterion and/or the predefined geographical zone; and
modifying the predefined criterion and/or the predefined geographical zone as requested in the communication in response to determining that the one of the one or more remote computing devices is authorized to make the modifications.

20. The computer program product of claim 15, the operations further comprising:
receiving, from the wearable device, biometric input and a request to unlock a locking mechanism that prevents the wearable device from being removed from the subject, wherein the wearable device includes a biometric sensor for capturing the biometric input;
determining whether the biometric input matches a stored biometric profile for a person that is authorized to remove the wearable device from the subject; and
transmitting a signal to deactivate the locking mechanism in response to determining that the received biometric input matches the stored biometric profile for the authorized person.
